(19) United States
(12) Patent Application Publication
(10) Pub. No.: US 2023/0277751 A1
(43) Pub. Date: Sep. 7, 2023

(54) UNIFORM INTERNAL DIAMETER FLUID DRAINAGE DEVICE AND SYSTEM

(71) Applicant: John D. Pavlus, San Antionio, TX (US)

(72) Inventor: John D. Pavlus, San Antionio, TX (US)

(73) Assignee: Oink Medical Group, LLC, San Antonio, TX (US)

(21) Appl. No.: 17/686,751

(22) Filed: Mar. 4, 2022

(51) Int. Cl. A61M 1/00; A61M 25/00

(52) U.S. Cl. CPC A61M 1/87; A61M 25/0097; A61M 25/0075; A61M 1/682; A61M 1/69

(57) ABSTRACT

A uniform internal diameter drainage system is disclosed. The system comprises a drainage catheter with large French drains and an internal diameter that progressively expands to a uniform internal diameter of the system. The catheter comprises a receiving port in fluid-tight communication with an engaging port of a hub, which comprises a first end, second end and flush port. The second end engages the catheter and the internal diameter of the catheter and the second end are substantially uniform. The first end comprises a receiving port that engages an engaging port of a drainage line and internal diameter of the drainage line and the first end of the system of the present invention are substantially uniform. A flush valve is disposed between the first end and second end of the present system to flush fluids from the system as needed.

### BACKGROUND

### 1. Field of the Invention

The present invention relates to systems and devices for draining fluids from patients and, more specifically, to a system and apparatus for draining bodily fluids from a patient wherein the internal diameter of the central hub of a drainage catheter system is substantially uniform throughout and of substantially uniform internal diameter at all or substantially all connection points within the system, namely between a drainage catheter and a central hub, and between the central hub and a drainage line in fluid connection to a drainage collection apparatus.

### 2. Description of the Related Art

Drainage of bodily fluids is necessary and desirable in various medical applications in order to reduce infection within the body of a patient and allow for the body to fight infection or reduce the mass effect a collection of fluids may have on the patient. Depending on the medical application and the location within the body cavity of the patient wherein the drainage of fluids is desired or needed, bodily fluids may be of differing viscosities. For instance, a catheter may be designed or adapted for a pneumothorax application, pericardial or plural drainage, hematoma drainage, biloma drainage or abdominal drainage.

Catheters and drainage systems have been utilized in the prior art to drain fluids from a patient in differing medical procedures and processes in treating a patient. The Cook pigtail catheter, which is known in the art, comprises a system wherein a catheter with a plurality of apertures or holes is placed within the desired location within the body cavity of a patient, and the line of the catheter extends outside of the body into a connection point that leads to a hub. It has a drainage line that continues on the side opposite the catheter to a gravity drainage bag, or suction device as is needed.

The connection points between the hub and the various lines of the Cook pigtail system comprise a lure lock system, as is known to one of ordinary skill in the art, wherein the internal diameter of the catheter line leading into the hub is of a smaller internal diameter than the receiving portion of the hub such that fluid will be forced into narrower internal diameters than either side lateral to the connection point of the lure lock. Furthermore, the lure lock system connecting the hub opposite the catheter line to the drainage line, (which leads to a gravity bag or suction apparatus) is similarly disposed such that the internal diameter of the hub is of a larger internal diameter sufficient to receive the smaller connection port of the drainage line. As a result, as fluid moves through the catheter into the hub and from the hub into the drainage line, pinch points are created through the system such that bodily fluid frequently gets stuck at various locations throughout the system. Furthermore, thick viscosity fluids pose a continued problem within current drainage systems, as the drains themselves and/or the connection points within the system will more easily clog as the viscosity of the fluid increases. In order to loosen the fluid within the pinch points to allow the system to continue to drain fluid from the patient, a flush port is required which also includes a lure lock connection mechanism to an external container with a fluid such as saline. A syringe or other suitable container containing saline is forcefully injected through the lure lock connection of the flush port to flush the fluids down the system and into the collection apparatus.

Catheters of the prior art comprise a string within the catheter that connects to the tip of the catheter so that when pulled, the catheter forms a loop or “pigtail.” The string extends within the distal end of the catheter, internal to the plurality of drainage holes, and is controlled from the hub to form the catheter loop internally so that the string maintains appropriate tension on the catheter to retain both the shape and location of the catheter within the patient. In addition to Cook, Boston Scientific, and Argon all comprise systems that require their connections to be universal to engage with a lure lock system in order to operate the drainage system. Moreover, the drains of the catheter of the prior art are often of insufficient size to accommodate the viscosity of the bodily fluids which they attempt to drain. Thal-Quick is a drain of larger internal diameter that contains a “lighthouse” connection in which the user typically must modify the system to a lure lock system in order to use a flush port or drainage receptacle.

The insufficient size of the drains of the catheter, coupled with the lure lock system of the prior art provides a system that is wrought with problems. First of all, the drainage of the bodily fluids through the drains of the catheter may not be sufficiently accomplished because of the clogging of the drains themselves, therefore causing the fluid which is desired to be drained from the patient to remain in the patient, or in efficiently drained. Second, the fluid that passes through the drains often clogs at the pinch points between the catheter line and the engagement points of the lure lock portions of the hubs, both in the catheter line and the drainage line. These results may require frequent flushing of the drainage system, or even replacement of drainage systems with larger drains, thus multiplying the number of procedures the patient must endure.

Accordingly, there is a need for an improved drainage system and apparatus to increase drainage and drainage efficiency within a patient as well as reduce the need for external saline or other flushing fluid to be imported into the system in order to flush the system where bodily fluids may clog at choke points or pinch points throughout the system. There is furthermore a need for an improved drainage system that utilizes drains that are sufficiently larger in size in order to accommodate thicker viscosity bodily fluids so as to prevent clogging within the patient's body cavity and to reduce the time needed to have a drain in place, effectually reducing the patient's hospital time or even reducing the number of procedures needed to replace or upsize a drain the drain is unable to be declogged.

### SUMMARY OF THE INVENTION

The present invention accomplishes the desirable aspects of improvement to existing drainage systems in a novel and unique way. The present invention comprises a complete system for fluid drainage from a patient wherein the internal diameter of the intricate parts and connections of the system are of a substantially uniform diameter such that a lure lock system is unnecessary for fluid flow or system connection, and fluid is allowed to drain substantially consistently throughout the system. With a substantially uniform internal diameter, the fluids of the body are allowed to flow consistently through the catheter line, hub, drainage line and into the collection apparatus without encountering pinch or choke points, which causes the fluid to clog. In one embodiment the connection to the collection apparatus has an internal diameter substantially uniform and identical to the internal diameter of the drainage line. With sufficiently reduced, or even eliminated clogging of the system, the need to flush the system with an external fluid such as saline is greatly reduced or even possibly eliminated. The system provides an advantage to the physician because the system drains the fluid more efficiently, and the need for manual labor to flush the system, thereby creating the inefficiency is reduced or eliminated.

The system of the present invention comprises a catheter with a plurality of apertures, which define drains along its distal end to be inserted within a patient that are significantly larger in size than typical drains of the prior art. In some embodiments, the bore size of the drains may range from a size of 8 French to 25 French. In other embodiments the bore size may range from 10 French to 20 French. In other embodiments, the drain may range in size from 12 to 18 French. In other embodiments, the bore size of the drains may range from 14 French to 16 French.

The internal diameter of the catheter progressively increases from the drains to a substantially uniform internal diameter leading external to the patient. External to the patient and at the end opposite the plurality of drains, the catheter of the present invention comprises a receiving port. The internal diameter of the catheter is substantially uniform from its greatest progressive expansion leading away from the drains to the receiving port

The hub of the present invention comprises a three-way system wherein a first end comprises a receiving port to receive a drain line of the present invention. A second end extends substantially opposite the first end and comprises an engaging port to engage the receiving port of the catheter. A flush port is mounted above the first end and second end and comprises a spring-loaded mechanism to engage an external fluid system such as a saline system (not shown) to flush the catheter and/or drainage lines if needed. The flush port may include a fluid-tight connection with a saline source via a lure lock connection system, or via a fluid-tight connection of a substantially uniform diameter with the first end and second end of the hub of the present system wherein flush port comprises a receiving port (not shown) and the saline source (not shown) comprises an engaging port to form a fluid-tight connection with the receiving port of the flush port substantially the same as the fluid tight connections between the catheter and hub, and the hub and the drain line. The receiving port of the first end engages an engaging port of a drain line. The connections between the drain line and the first end provide a fluid-tight connection wherein the internal diameter of the drain line and the internal diameter of the first end of hub of are substantially identical.

The receiving port of the catheter line and the engaging port of the second end provide a fluid-tight connection such that the internal diameter of the catheter, and the internal diameter of the second end are substantially identical/uniform. Between the first and second end of the hub of the present invention, a valve as is known to one of ordinary skill in the art is disposed, and the valve is operated manually via a knob located external to the hub and extending therethrough to operate the valve between the first and second end of the hub. The valve provides for directional control of the flow of fluids during use, and in the event of the need to flush the present system. Alternatively, the knob of the valve could be located along the top portion of the flush port or other locations along the hub where it can pass through to the valve to operate the valve to control the direction of flow of fluids.

The drain line of the present invention engages a drainage receptacle which may be selected from a group consisting of a drainage gravity bag, an according suction, and/or a suction bulb or suction syringe. However other drainage receptacles known in the art may be used. The fluid-tight connections between the first end of the hub and the drain line, as well as the fluid-tight connection between the second end and the catheter line are provided by a snap and release mechanism on each end, both attached to the receiving port of the hub and catheter line, respectively.

For insertion of the catheter within the patient, the present invention comprises a stiffener or stylet that engages the snap and release mechanism of the receiving port of the catheter. The stylet comprises a chamfered surface that engages the receiving port of the catheter, and an annular ring that engages the snap and release mechanism to form a fluid tight connection. A guide wire (not shown) is placed through this stylet, and the guide wire may be made of soft or hard materials. The guide wire is utilized to guide the end of the catheter comprising the drains into the patient to the desired location and maintains the curled or “pigtail” shape of the catheter along its distal end.

The feature or features of one embodiment can be applied to other embodiments, even though not described, or illustrated, unless expressly prohibited by this disclosure or the nature of the embodiments. Some details associated with the embodiments described above and others are described below.

### BRIEF DESCRIPTION OF THE DRAWINGS

The following drawings illustrated by way of example and not limitation. For the sake of brevity and clarity, every feature of a given structure is not always labeled in every figure in which that feature appears. Identical reference numbers do not necessarily indicate an identical structure. Rather, the same reference number can be used to indicate a similar feature or feature with similar functionality, as can non-identical reference numbers.

FIG. 1 is a side view of the system of the present invention comprising the catheter and fluid-tight connection with the hub, and the drain line in fluid-tight connection with the hub on one end, and a fluid receiving device on the opposite end.

FIG. 2 is a top view of the system of the present invention.

FIG. 3 is a side view of the present invention showing the stylet and connection apparatus for guiding a guide wire (not shown) within the catheter to place the catheter within the patient (not shown).

FIG. 4 is a perspective view of the snap and release mechanism of the receiving port of the first end of the hub and the engaging port of the drainage line of the present invention.

FIG. 4a is a perspective view of the snap and release mechanism of the receiving port of the catheter and the engaging port of the second end of the hub.

FIG. 5 is an exploded view of the catheter, hub and drainage line of the present invention.

### DETAILED DESCRIPTION

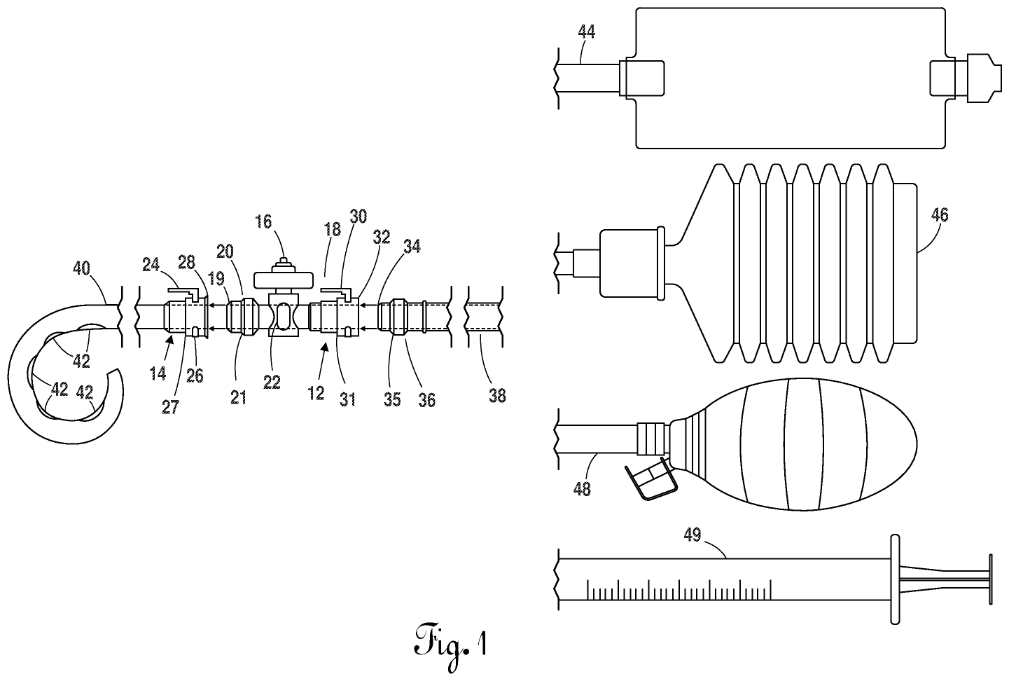

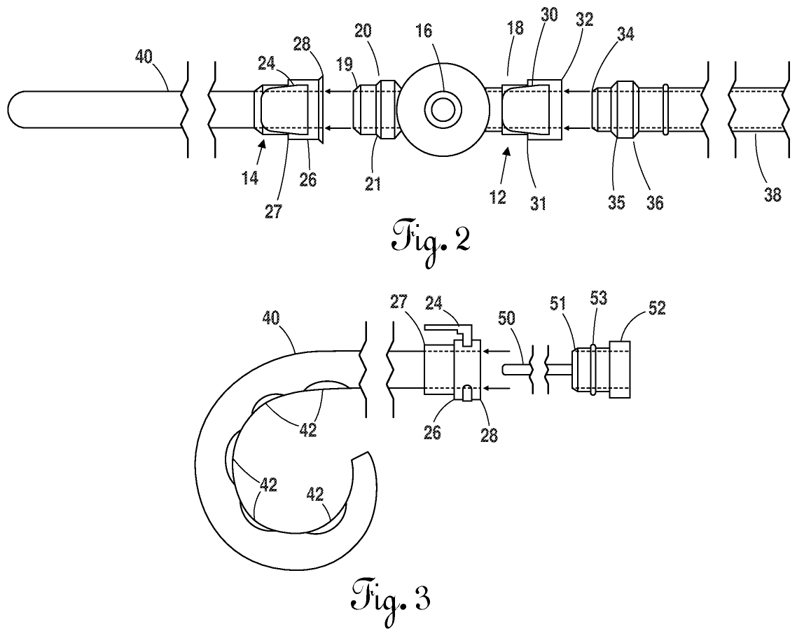

Referring to FIGS. 1 through 5, the system and apparatus for uniform internal diameter drainage device 10 is disclosed. The present invention comprises a first end 12, a second end 14 and a flush port 16, defining a hub, a catheter 40 in fluid-tight connection with second end 14, and a drainage line 38 in fluid-tight connection with the first end 12. The drainage line 38, along the end portion opposite the end that engages first end 12 is in fluid-tight connection with a fluid receptacle. The fluid receptacle could be any fluid receptacle known by one of ordinary skill in the art and may be selected from the group consisting of a drainage gravity bag 44, an accordion suction 46, a suction bulb 48, or a suction syringe 49 (see FIG. 1). In one embodiment the connection to the drainage gravity bag 44, an accordion suction 46, a suction bulb 48, or a suction syringe 49 have an internal diameter at the connection to drainage line 38 substantially uniform and identical to the internal diameter of the drainage line 38.

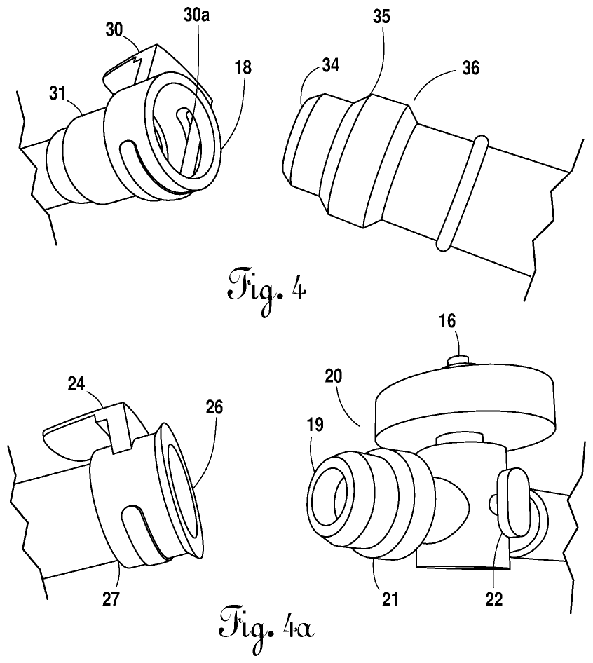

Referring to FIG. 3, the catheter 40 may engage a guide wire support apparatus 52, which comprises a stylet 50 which receives a guide wire (not shown) which extends through the catheter 40, into the distal end where the apertures defining the large French drains 42 are disposed. The guide wire support apparatus 52 comprises an chamfered surface 51 which engages a receiving port 26 of the catheter 40, and an annular ring 53 which engages with snap and release mechanism 24 to form a fluid-tight connection. The guide wire extends within catheter 40 and terminates attached to and within the distal end of catheter 40 and is guided through the stylet 50 and is manipulated by the physician (not shown) to place the catheter 40 within the patient at the desired location while maintaining the substantially curved or pigtail shape of catheter 40. The stylet 50 engages with in the receiving port 26 of the catheter 40 and engages with the snap and release mechanism 24 of the receiving port 26. Once placed within the patient at the desired location, snap and release mechanism 24 is depressed, causing annular ring 53 disengage with receiving port 26, and the guide wire support apparatus 52, including the stylet for the guide wire, are removed, leaving the guide wire in place within the catheter 40.

Referring to FIGS. 1, 2, 4, 4a and 5 the first end 12 comprises a receiving port 18. The receiving port 18 comprises, along its distal end, a snap and release mechanism 30. The snap and release mechanism 30 extends partially within the internal diameter of the receiving port 18 long and end substantially opposite the releasing handle of the snap and release mechanism 30. The first end 12 extends inwardly within the hub to the valve 22 and is fluid-tight communication with the flush port 16.

The drainage line 38 comprises an engaging port 36. Along its distal end, the engaging port 36 comprises a chamfered connection surface 34 which allows for ease of insertion within the receiving port 18 of the first end 12. Behind the chamfered connection 34, an annular ring 35 is disposed, which extends annularly outward and external to the external diameter of the engaging port 36. Internal to the receiving port 18 of the first end 12, a shoulder 31 rests within the internal diameter. The shoulder 31 engages chamfered surface 34 to provide a hard stop, preventing the drainage line 38 from moving within the hub, and specifically within the first end 12.

The annular ring 35 engages the internal portion of the snap and release mechanism 30 to snap engaging port 36 within the receiving port 18. The annular ring 35 and the chamfered surface 34 provide a seated ring relationship with the internal diameter of first end 12 and engages within the internal diameter of the first end 12 to create a fluid-tight seal. The internal diameter of the drainage line 38 is of a substantially uniform diameter, and substantially identical internal diameter with first end 12. The internal diameter, from the drain line 38, through engaging port 36 is substantially uniform and identical to the internal diameter of first end 12, through receiving port 18, and through first end 12 to the valve 22. The opening (not shown) in the valve 22 has a substantially identical internal diameter to the internal diameter of first end 12.

Referring to catheter 40, a plurality of large French drains 42 are disposed along the distal end, and the catheter 40 creates a curling shape as it is placed within the patient. The internal diameter of the distal end of catheter 40 along large French drains 42 is smaller than the internal diameter of second end 14. However, moving from the distal end of catheter 40, the internal diameter of catheter 40 progressively increases to the point that the internal diameter of catheter 40 past large French drains 42 is of substantially identical diameter to second end 14. Therefore, so long as fluid is of sufficient viscosity to pass through large French drains 42 and internally to the point of the substantial uniform internal diameter of the catheter 40, the fluid will be able to flow through the system 10 of the present invention without encountering pinch points or smaller areas of internal diameter which would aid or promote clogging. In some embodiments, the bore size of the drains 42 may range from a size of 8 French to 25 French. In other embodiments the bore size of the drains 42 may range from 10 French to 20 French. In other embodiments, the drain 42 may range in size from 12 to 18 French. In other embodiments, the bore size of the drains may range from 14 French to 16 French.

Referring to FIGS. 1,4a and 5, along the proximal end, opposite of the distal end of catheter 40, a receiving port 26 engages the engaging port 20 of the second end 14. Second end 14 of the hub comprises an engaging port 20. Along its distal end, the engaging port 20 comprises a chamfered connection surface 19 which allows for ease of insertion within the receiving port 26 of the catheter 40. Behind the chamfered connection 19, an annular ring 21 is disposed extending annularly outward and external to the external diameter of the engaging port 20. Internal to the receiving port 24 of the catheter 40, a shoulder 27 rests within the internal diameter. The shoulder 27 engages chamfered surface 19 to provide a hard stop, preventing the second end 14 from moving within the catheter 40.

The annular ring 21 engages the internal portion of the snap and release mechanism 24 to snap engaging port 20 within the receiving port 26. The annular ring 21 and the chamfered surface 21 provide a seated ring relationship with the internal diameter of receiving port 26 and engages within the internal diameter of the receiving port 26 to create a fluid-tight seal. The internal diameter of the catheter 40 is of a substantially uniform diameter, and substantially identical internal diameter with second end 14. The internal diameter, from the receiving port 26 of catheter 40 through engaging port 20 of second end 14, is substantially uniform and identical to the valve 22. The opening (not shown) in the valve 22 has a substantially identical internal diameter to the internal diameter of second end 14.

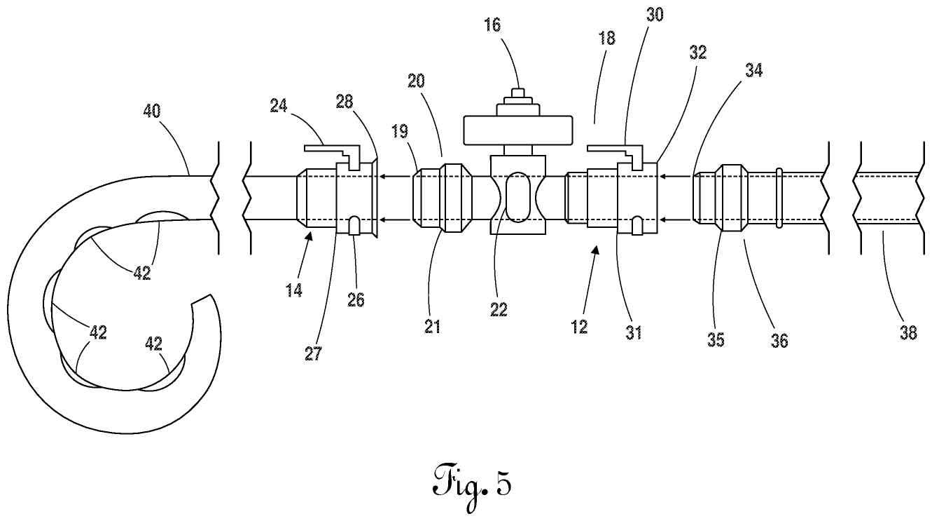

Referring to FIGS. 4, and 4a the fluid-tight connections of the present invention are shown. Referring to FIG. 4, to release the fluid-tight connection and disengage first end 12 from drainage line 38, snap and release mechanism 30 is depressed to bend the engagement portion 30a thereof outward such that it disengages from annular ring 35. Drainage line 38 is pulled outward from first end 12, and the annular ring 35 slides past engagement portion 30a of snap and release mechanism 30 to disengage the drainage line 38 from first end 12.

Referring to FIG. 4a, to release the fluid-tight connection and disengage second end 14 from catheter 40, snap and release mechanism 24 is depressed to bend the engagement portion (not shown) thereof outward such that it disengages from annular ring 21. Catheter 40 is pulled outward from second end 14, and the annular ring 21 slides past snap and release mechanism 24 to disengage the catheter 40 from second end 14.

Flush port 16 comprises a spring-loaded opening that is designed to engage in a lure lock fashion (not shown) with an external fluid source (not shown). The external fluid source may be contained in any suitable container such as a syringe or other suitable container that may engage with flush port 16 to flush the system as needed in the event that the body fluids become of such a viscosity as to clog the system. However, it is also contemplated that a flush port 16 could be designed such that it has a uniform internal diameter and external diameter to be a fluid-tight connection with an external fluid flushing source in the same manner and connection in which the drainage line 38 is in fluid-tight connection with the hub and the catheter 40 is in fluid-tight connection with the hub of the present system 10.

The above specification and examples provide a complete description of the structure and use of the illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods, systems and apparatuses are not intended to be limited to particular forms as disclosed herein. Rather they include all modifications and alternatives falling within the scope of the claims, and the embodiments other than the ones shown can include some or all of the features of the depicted embodiment. For example, elements can be omitted or combined as a unitary structure, and/or connections can be substituted. Further, where appropriate, aspects of any of the examples described above can be combined with aspects of any other examples described to form further examples having comparable or different properties and/or functions and addressing the same or different problems. Similarly, it will be understood that the benefits and advantages described above can relate to one embodiment or can relate to several embodiments.

The claims are not intended to include, and should not be interpreted to include, means plus or step plus function limitations, unless such a limitation is explicitly resided in a given claim using the phrase “means for” or “step for” respectively.

We claim:

1. A uniform internal diameter drainage device comprising:
a multi-direction hub comprising:
a first end comprising a predetermined internal diameter and terminating in a first receiving port;
a second end comprising said predetermined internal diameter and terminating in an engaging port;
a valve between said first end and said second end, said valve comprising a directional flow apparatus to operate said valve to determine a direction of flow; and
a flush port adjacent said valve and in fluid communication with said first end and said second end within said predetermined internal diameter;
wherein said predetermined internal diameter of said first end and said second end comprise a substantially uniform internal diameter.

2. The uniform internal diameter drainage device as set forth in claim 1 further comprising a catheter, said catheter comprising a receiving port in fluid-tight communication with said engaging port of said second end of said drainage device, and terminating along a portion opposite said receiving port in a plurality of drainage apertures; and
wherein said catheter has a predetermined internal diameter substantially uniform to said predetermined internal diameter of said first end and said second end of said drainage device

3. The uniform internal diameter drainage device as set forth in claim 2 comprising a fluid drainage line comprising an engaging port in fluid-tight communication with said receiving port of said drainage device, and in fluid-tight communication along an end opposite said engaging port with a drainage receptacle; and
wherein said drainage line has a predetermined internal diameter substantially uniform to said predetermined internal diameter of said first end and said second end of said drainage device.

4. The uniform internal diameter drainage device as set forth in claim 3 wherein:
said receiving port of said first end of said drainage device comprises an externally annular snap and release mechanism, an internal annular recess and an annular shoulder;
said engaging port of said first end comprises a chamfered connection surface on its distal end engaging said annular shoulder, and an annular ring engaging said annular recess to form said fluid-tight connection.

5. The uniform internal diameter drainage device as set forth in claim 4 wherein:
said receiving port of said second end of said drainage device comprises a externally annular snap and release mechanism, an internal annular recess and an annular shoulder; and
said engaging port of said second end comprises a chamfered connection surface on its distal end engaging said annular shoulder, and an annular ring engaging said annular recess to form said fluid-tight connection.

6. The uniform internal diameter drainage device as set forth in claim 5 wherein said flush port is a spring loaded flush port and receives an external saline source for flushing said drainage device.

7. The uniform internal diameter drainage device as set forth in claim 6 wherein said drainage receptacle is selected from a drainage bag, an accordion suction and a suction bulb.

8. The uniform internal diameter drainage device as set forth in claim 7 wherein said apertures of said catheter are of substantially uniform size and shape.

9. The uniform internal diameter drainage device as set forth in claim 8 wherein said flush port comprises an internal diameter substantially uniform said internal diameter of said first end and said second end.

10. The uniform internal diameter drainage device as set forth in claim 9 wherein said flush port comprises an internal diameter different than said internal diameter of said first end and said second end.

11. A system for reducing fluid blockage in a catheter draining fluid from a patient comprising:
a catheter for insertion into a patient to drain said fluid therefrom, said catheter comprising a predetermined internal diameter, a plurality of drains which are substantially uniform, and terminating opposite said plurality of drains in a receiving port;
a drainage line comprising an engaging port along a first end and in fluid communication with a drainage receptacle adjacent its second end, said drainage line comprising a predetermined internal diameter substantially uniform with said internal diameter of said catheter;
a central hub comprising:
a first end comprising a receiving port and connected to said drainage line by engaging said engaging port of said drainage line to create a fluid-tight connection;
a second end opposite said first end and comprising an engaging port connected to said receiving port of said catheter to create a fluid-tight connection;
a valve between said first end and said second end, said valve comprising a directional flow apparatus to operate said valve to determine a direction of flow; and
a flush port adjacent said valve and in fluid-tight communication with said first end and said second end.

12. The system for reducing fluid blockage in a catheter draining fluid from a patient as set forth in claim 11 wherein:
said receiving port of said first end of said drainage device comprises an externally annular snap and release mechanism, an internal annular recess and an annular shoulder;
said engaging port of said first end comprises a chamfered connection surface on its distal end engaging said annular shoulder, and an annular ring engaging said annular recess to form said fluid-tight connection.

13. The system for reducing fluid blockage in a catheter draining fluid from a patient as set forth in claim 12 wherein:
said receiving port of said second end of said drainage device comprises a externally annular snap and release mechanism, an internal annular recess and an annular shoulder; and
said engaging port of said second end comprises a chamfered connection surface on its distal end engaging said annular shoulder, and an annular ring engaging said annular recess to form said fluid-tight connection.

14. The system for reducing fluid blockage in a catheter draining fluid from a patient as set forth in claim 13 said flush port is a spring loaded flush port and receives an external saline source for flushing said drainage device.

15. The system for reducing fluid blockage in a catheter draining fluid from a patient as set forth in claim 14 said drainage receptacle is selected from a drainage bag, an accordion suction and a suction bulb.

16. The system for reducing fluid blockage in a catheter draining fluid from a patient as set forth in claim 15 wherein said flush port comprises an internal diameter substantially uniform said internal diameter of said first end and said second end.

17. The system for reducing fluid blockage in a catheter draining fluid from a patient as set forth in claim 16 wherein said flush port comprises an internal diameter different than said internal diameter of said first end and said second end and is attachable via leur lock to an external fluid source to flush said system.

18. A three direction catheter hub for draining fluids from a patient comprising:
a first end comprising a first receiving port with a releasing mechanism, said first receiving port attachable to an engaging port of a catheter line leading to a drainage apparatus;
a second end comprising an engaging port attachable to a receiving port of a catheter line leading to a drainage site within said patient;
a valve between said first end and said second end, said valve comprising a directional flow apparatus to operate said valve to determine a direction of flow; and
a flush port comprising said predetermined internal diameter adjacent said valve and in fluid communication with said valve;
wherein said first end, said second end, and said flush port comprise a substantially uniform predetermined interior diameter.
